(19) United States
(12) Patent Application Publication
(10) Pub. No.: US 2002/0150547 A1
(43) Pub. Date: Oct. 17, 2002

(54) Composition of hair growth facilitator

(75) Inventors: Kye Ho Lee, Seoul (KR); Ki Tae Han, Suwon (KR); Ree Won Joo, Koyang-shi (KR); Min Gyu Cho, Seoul (KR); Young Soo Lee, Seoul (KR); Chang Ho Jeoung, Seoul (KR); Min Su Park, Seoul (KR); Sun Il Han, Seoul (KR); Ji Young Oh, Seoul (KR); Jun Gu Kang, Kunpo-shi (KR)

Correspondence Address: FINNEGAN, HENDERSON, FARABOW, GARRETT &, DUNNER LLP, 1300 I STREET, NW, WASHINGTON, DC, 20005, US

(73) Assignee: STC NARA CO., LTD.

(21) Appl. No.: 09/897,482

(22) Filed: Jul. 3, 2001

(30) Foreign Application Priority Data
Dec. 26, 2000 (KR) 2000-81935

(51) Int. Cl. A61K 7/06; A61K 35/78

(52) U.S. Cl. 424/74; 424/770; 424/773

(57) ABSTRACT

The present invention relates to a composition of a hair growth facilitator comprising camphor extract, morus bark root extract, new shoot extract of a pine tree, and fibrous biopolymer produced by silkworm cocoons. By having a superior hair growth effect without any known side effects or toxicities, the present invention can resolve the drawbacks present in the conventional compositions of hair growth facilitators and thus can be safely applied to various hair-related products such as hair tonic, hair lotion, hair cream, hair shampoo and hair conditioner.

### BACKGROUND OF THE INVENTION 1. Field of the Invention

The present invention relates to a composition of a hair growth facilitator and, more particularly, to a composition of a hair growth facilitator which comprises a blood circulation promoter and a hair follicle cell activator, wherein said blood circulation promoter consists of camphor extract, morus bark root extract, and new shoot extract of a pine tree, and said follicle cell activator consists of a fibrous biopolymer produced by silkworm cocoons. 2. Description of the Related Arts

Humans in general have approximately 0.1-0.15 million hairs on the head, and each hair undergoes a biological hair cycle consisting of anagen, catagen and telogen in this order and finally falls off. This cycle of a hair is periodically repeated at the intervals of 3-6 years and approximately 50-200 hairs are fallen off daily on the average. Here, when the relative percentage of hairs in the anagen becomes lower while the corresponding percentages of hairs in the catagen and the telogen of the hair cycle are increased, it results in an abnormal marked decrease in the number of total hairs on the head and is generally referred to as Alopecia.

Numerous reasons have been known to cause Alopecia, for example, hypogasia of hair follicle, hypogasia of metabolism in hair follicle/hair root, hypogasia of scalp, hypergasia of the flexibility of scalp, local dysfunction of blood circulation due to a decrease of blood flow in peripheral blood vessels located at the head hypoderm, malnutrition, stress, side effects resulted from medications, hereditary defects and the like. Also, there have been introduced various ways to nourish hairs as well as to prevent and therapeutically treat the alopecia, for example, by administering blood circulation promoters, anti-male sex hormones, anti-seborrheic agents, keratolytic agents, anti-inflammatory disinfectants, various amino acids and vitamins for the nourishment of hair follicle and the activation of enzyme activities involved therein, and local stimulation of scalp, however, none of them have been shown to be effective in performing their designed purposes.

### SUMMARY OF THE INVENTION

To remedy the inefficiencies of the conventional methods and pharmaceutical agents in promoting hair growth as well as in preventing and treating the symptoms of abnormal hair loss, the inventors of the present invention conducted enormous researches and finally completed the present invention by developing a composition having an excellent effect on hair growth without any side effects or toxicities comprising a blood circulation facilitator and a hair follicle cell activator, wherein said blood circulation facilitator consists of camphor extract, the morus bark root extract, and new shoot extract of a pine tree, whereas said follicle cell activator consists of a fibrous biopolymer produced by silkworm cocoons.

### Brief Description of Drawings

FIG. 1 shows three different pictures representing the effect of a given composition on hair growth, wherein each composition is prepared in accordance with Example 1, Comparative Example 1, and Comparative Example 2, respectively, and applied independently to each experimental mice group.

### DETAILED DESCRIPTION OF THE INVENTION

The present invention relates to a composition of a hair growth facilitator comprising a blood circulation promoter and a hair follicle cell activator, wherein said blood circulation promoter consists of 0.01-10% by wt of camphor extract, 0.1-30% by wt of morus bark root extract, and 0.1-20% by wt of new shoot extract of a pine tree, whereas said follicle cell activator consists of 0.01-30% by wt of a fibrous biopolymer produced by silkworm cocoons, which does not show any known side effects or toxicities but has a superior facilitator of hair growth.

The present invention is explained in more detail as set forth hereunder.

First, camphor extract, root of morus bark extract, and new shoot extract of a pine tree are prepared as follows.

The root, stalks and leaves of camphor; root of morus bark; and new shoots of a pine tree are first selected under a strict examination and then pretreated by subjecting them independently to steps of washing, mincing, sterilization, and drying in this order, immersing them into equal amount of water or ethanol solution or the mixture of water and ethanol for 30 min-48 hr kept at 20-120° C., and then their extracts were obtained, respectively. Thus obtained extracts are then filtered, and 0.01-10% by wt of camphor extract, 0.1-30 % by wt morus bark root extract, and 0.1-20% by wt of new shoot extract of a pine tree, are combined with 0.01-30% by wt of a biopolymer taking on a strong fibrous glossiness produced by either silkworm cocoons or moth larvae called Bombyx mori, which comprise the active ingredients of the composition of the hair growth facilitator according to the present invention.

The morus bark root extract as an ingredient for the composition according to the present invention can be obtained by extracting the bark and cork of a mulberry tree or a plant of the same genus(Moraceae) of the mulberry tree by using water or ethanol. This extract contains flavonoid, tannin, scopoletin, and a few insect hormones such as ecdysterone and incosterone as active ingredients. This morus bark root extract has various pharmaceutical actions which include diuresis, antihypertensive activity, antipyretic action, antitussive activity, beriberi, etc. Further, the morus bark root extract has various cosmetic effects which include skin whitening, anti-inflammatory activity, hair nourishment, inhibition of lipid peroxide, inhibition of dandruffs, improvement of blood circulation, and activation of epidermal metabolism. The preferred amount of morus bark root extract is 0.1-30% by wt. If the amount is less than 0.1% by wt, the desired effect cannot be performed while if it is more than 30% by wt, it would result in excessive tannin content thus becoming not desirable.

The new shoot extract of a pine tree as an ingredient for the composition of a hair growth facilitator according to the present invention can be obtained by extraction of new shoots of a pine tree(red pine) after adding equal amount of a mixture of water and ethanol, and this extract contains quercetin glucoside and kaempferol glucoside as active ingredients. The pharmaceutical actions of this extract include effects of anti-inflammation and antioxidation. The preferred amount of new shoot extract of a pine tree is 0.1-20% by wt. If the amount is less than 0.1% by wt, the desired inflammatory effect becomes deteriorated while if it is more than 20% by wt, the effective balance with other active ingredients cannot be maintained.

The extract of camphor as an ingredient for the composition according to the present invention can be obtained by extraction of root, stalks, and leaves of Laurus camphora of more than 50 years of age, which are widespread in Japan and Sumatra in Indonesia, by means of water or ethanol. The pharmaceutical actions of this extract include the improvement of blood circulation and the anti-inflammatory effect The preferred amount of this extract is 0.01-10% by wt. If the amount is less than 0.01% by wt, the desired inflammatory effect becomes deteriorated while if it is over 10% by wt, the effective balance with other active ingredients cannot be maintained and can also generate unpleasant odors.

The extract of a biopolymer as an ingredient for the composition of a hair growth facilitator according to the present invention can be obtained by extraction of either silkworm cocoons or moth larvae called Bombyx mori. The biopolymer is a silk protein which has a similar structure as keratin in hairs and it can also affect the activation of follicle cells of a hair. The preferred amount of this extract is 0.01-30% by wt. If the amount is less than 0.01% by wt, the desired activation of hair follicle cells cannot be achieved while if it is more than 30% by wt, the effective balance with other active ingredients cannot be maintained and also the resulting preparation will remain unstable.

There are no certain limitations on bases that can be used in preparing a composition of a hair growth facilitator according to the present invention as long as it can improve the cosmetic effects and control the concentrations. For example, the ustable bases include purified water, monovalent alcohols, multivalent alcohols, high grade alcohols, oils and fats, paraffins, emulsifiers, and solubilizers. The preferred amount of the bases when used either alone or as a combined mixture of the above bases is 70-95% by wt and this is determined based on the amount of the composition of the hair growth facilitator being used.

The composition of a hair growth facilitator in the present invention comprises camphor extract, morus bark root extract, and new shoot extract of a pine tree, and a fibrous biopolymer as active ingredients, and other ingredients generally used in conventional hair growth facilitators such as cayenne pepper tincture, tocopherol acetate, nicotinic-acid benzyl, ginkgo leaf extract, hinokitiol for stimulation of hair root and hair cell, nicotinic-acid amide, L-menthol, extract of polygonum multiflorum Thunb, propolis extract or piroctoneolamine for keratolysis, salicylic acid, extract of Luffa cylindrical, a luffa, ethanol, and other humectants can be also used as additional ingredients along with said active ingredients.

The concentrations of the ingredients such as L-menthol, extract of polygonum multiflorum Thunb, propolis extract, ethanol, luffar extract (Luffa cylindrical), gingko leaf extract, etc. used as ingredients for the facilitator of hair growth in this invention are not particularly limited as long as they do not affect the mode of final preparations nor the effects of the active ingredients contained in said preparations, however, for the purposes of human safety and the stability of preparations, it is preferred that the amount of L-menthol be 0.01-1.0% by wt; extract of polygonum multiflorum Thunb, propolis extract, luffar extract, and ginkgo leaf extract be all 0.01-10% by wt, respectively; and ethanol 1-50% by wt.

The composition of a hair growth facilitator prepared according to the present invention can be used in manufacturing cosmetic hair products such as hair tonics, hair creams, hair shampoos and hair conditioners and they are preferred to contain 5-30% by wt of said composition.

This invention is explained in much greater detail by the following examples, but they should not be construed as limiting the scope of the present invention.

### EXAMPLE 1 AND COMPARATIVE EXAMPLES 1-4

### The Effects of Hair Growth Facilitators in Experimental Mice

The mice used in these experiments were C3H mice which are frequently used in observing the effects of a given composition on hair growth because the onset of hair growth in C3H mice can be easily detected by visual observations due to their color change from pink to blue upon the initiation of hair growth.

In these experiments, hairs on the back of each mouse were removed by using an electric shaver. Then, the weight of an individual mouse was measured and all the mice were divided into a few groups so that each group can consist of five mice having a similar weight distribution. The mice were then allowed to adapt to a new environment for a day and were sprayed with liquid hair growth facilitators, which were prepared according to the compositions in the Example 1, Comparative Examples 1-4 shown in the table 1, on the back of mice where hairs were removed three times/day at 9 a.m., 1 p.m., and 6 p.m. starting from the second day. The amount of each hair growth facilitator composition applied on mice was approximately 0.3 cc and the area where hair growth was promoted was observed via an image analyzer, and the level of hair growth and the distribution of hair growth were examined via photo-pictures taken. The facilitators were administered for 25 days.

TABLE 1

| Ingredient | Com. Ex. | Com. Ex. | Com. Ex. | Com. Ex. | Ex. |
|---|---|---|---|---|---|
| | 1 | 2 | 3 | 4 | 1 |
| Ethanol | 45.0 | 45.0 | 45.0 | 45.0 | 45.0 |
| Propylene | — | 2.0 | 2.0 | 2.0 | 2.0 |
| Glycol | | | | | |
| Luffar | — | 1.0 | — | — | 1.0 |
| Extract | | | | | |
| L-menthol | — | 0.2 | 0.2 | 0.2 | 0.2 |
| Extract of | — | 1.0 | 1.0 | 1.0 | 1.0 |
| polygonum | | | | | |
| multiflorum | | | | | |
| Thunb | | | | | |
| Propolis | — | 1.5 | 1.5 | 1.5 | 1.5 |
| Extract | | | | | |
| Gingko Leaf | — | 2.0 | 2.0 | — | 2.0 |
| Extract | | | | | |
| Morus Bark | — | — | — | — | 1.0 |
| Root Extract | | | | | |
| New Shoot | — | — | — | — | 2.0 |
| Extract of | | | | | |
| a Pine tree | | | | | |
| Camphor | — | — | — | — | 0.2 |
| Extract | | | | | |
| Biopolymer | — | — | — | — | 0.1 |
| Tincture, | Adequate | Adequate | Adequate | Adequate | Adequate |
| Flavor | | | | | |

| | |
|---|---|
| Purified | Add to 100% by wt |
| Water | |

Com. Ex.: Comparative Example
Ex.: Example

As shown in table 1, the composition of a hair growth facilitator prepared according to the comparative example 2 consists of other supplemental ingredients which exclude morus bark root extract, new shoot extract of a pine tree, camphor extract, and biopolymer. The composition of a hair growth facilitator prepared according to the comparative example 3 consists of other supplemental ingredients which exclude morus bark root extract, new shoot extract of a pine tree, camphor extract, a biopolymer and luffar extract. The composition of a hair growth facilitator prepared according to the comparative example 4 consists of other supplemental ingredients which exclude morus bark root extract, new shoot extract of a pine tree, camphor extract, a biopolymer, luffar extract and ginkgo leaf extract. The composition of hair growth facilitator prepared according to the example 1 consists of supplemental ingredients as well as all the active ingredients listed in the above table 1 and was administered to the experimental mice in the form of a liquid hair growth facilitator.

The ratio between the area where new hair growth was observed and the area where the original body hairs were removed by an electric shaver was calculated and the results were classified into five different levels for evaluation purpose as shown in the following table 2.

TABLE 2

| Level | Area for New Hair Growth (%) |
|---|---|
| 4 | >70 |
| 3 | 50˜70 |
| 2 | 30˜50 |
| 1 | <30 |
| 0 | No Hair Growth |

The following table 3 shows the comparative data of the new hair growth area among example 1 and comparative examples 1-4.

TABLE 3

| | Com. | | | | |
|---|---|---|---|---|---|
| Classification | Ex. 1 | Com. Ex. 2 | Com. Ex. 3 | Com. Ex. 4 | Ex. 1 |
| Ratio of Area | 1.6 | 2.1 | 1.8 | 1.7 | 4.3 |

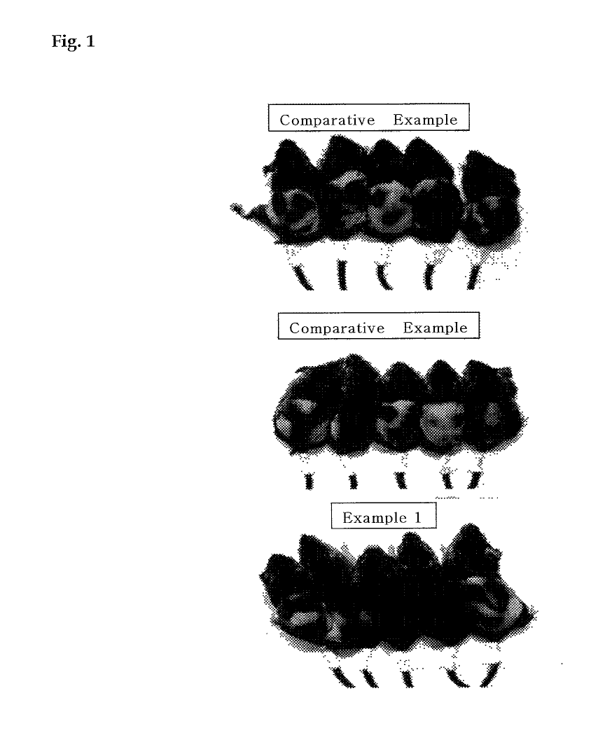

From the above table 3, it is apparent that the composition prepared according to the example 1, wherein said composition contains morus bark root extract, new shoot extract of a pine tree, camphor extract, and a biopolymer, has a much higher rate of new hair growth as compared to that of comparative example 1, approximately by a factor of more than 2.5, and the factor was more than 2.0 as compared to that of comparative example 2, wherein said composition contains only supplemental ingredients and is exclusive the active ingredients listed in the above table 1. The pictures that depict the results of the experiments of various compositions applied in the experimental mice are shown in the FIG. 1.

### EXAMPLE 2

Based on the result of example 1, various sets of compositions were prepared as shown in the following table 4.

TABLE 4

| Classification | Hair | Hair | Hair | Hair | Hair |
|---|---|---|---|---|---|
| (wt %) | tonic | shampoo | conditioner | lotion | cream |
| Ethanol | 45.0 | — | — | — | — |
| Propylene Glycol | 2.0 | 2.0 | 2.0 | 2.0 | 2.0 |
| Luffar Extract | 0.1 | 0.1 | 0.1 | 0.1 | 0.1 |
| L-menthol | 0.2 | 0.2 | 0.2 | 0.2 | 0.2 |
| Extract of | 1.0 | 1.0 | 1.0 | 1.0 | 1.0 |
| polygonum | | | | | |
| multfflo rum Thunb | | | | | |
| Propolis Extract | 1.5 | 1.5 | 1.5 | 1.5 | 1.5 |
| Gingko Leaf Extract | 2.0 | 2.0 | 2.0 | 2.0 | 2.0 |
| Morus Bark Root | 1.0 | 1.0 | 1.0 | 2.0 | 2.0 |
| Extract | | | | | |
| New Shoot Extract of | 2.0 | 1.0 | 1.0 | 2.0 | 2.0 |
| a Pine tree | | | | | |
| Camphor Extract | 0.2 | 0.1 | 0.1 | 0.2 | 0.2 |
| Biopolymer | 0.1 | 0,1 | 0.1 | 0.2 | 0.3 |
| POE Sodium Lauryl | — | 40.0 | — | — | — |
| Sulfate (30%) | | | | | |
| Palm oil Fatty acid | — | 3.0 | — | — | — |
| Diethanol amide | | | | | |
| p-oxy benzoic ester | — | 0.3 | — | — | |
| Tocopherol acetate | — | 0.1 | — | — | — |
| Cetanol | — | — | 3.0 | 2.0 | — |
| Self-emulsifying | | | | | |
| glycerol | — | — | 2.0 | 2.0 | — |
| monostearate | | | | | |
| Stearyl dimethyl | 8.0 | | | | |
| benzyl ammonium | — | — | | | |
| chloride (25%) | | | | | |
| Sorbitan | — | — | — | 1.0 | — |
| monostearate | | | | | |
| Vaseline | — | — | — | 2.0 | — |
| Liquid paraffin | — | — | — | 6.0 | 1.0 |
| Squalene | — | — | — | 4.0 | 2.0 |
| Carboxy vinyl | — | — | — | 0.2 | 0.3 |
| polymer | 0.2 | 0.3 | | | |
| Triethanol-amine | — | 1.0 | | | |
| Polysorbates 60 | — | — | — | — | 1.0 |
| Sorbitan sesquioleate | — | — | — | — | 3.0 |
| Tincture, Flavor | Adequate | Adequate | Adequate | Adequate | Adequate |

| | |
|---|---|
| Purified Water | Add to 100% by wt |

As mentioned above, the present invention provides a composition of a hair growth facilitator which has a superior effect on hair growth without any side-effects or toxicities and thus can be safely applied to various hair-related products such as hair tonic, hair lotion, hair cream, hair shampoo, and hair conditioner.

What is claimed is:

1. A composition of a hair growth facilitator, wherein said composition comprises 0.1-30% by wt of morus bark root extract, 0.1-20% by wt of new shoot extract of a pine tree, 0.01-10% by wt of camphor extract, and 0.01-30% by wt of biopolymer.

2. The composition of a hair growth facilitator according to claim 1, wherein said composition can be used in hair-related products such as a hair tonic, a hair lotion, a hair cream, a hair shampoo, and a hair conditioner.
